(19) United States
(12) Patent Application Publication
(10) Pub. No.: US 2020/0022600 A1
(43) Pub. Date: Jan. 23, 2020

(54) WIRELESS BIOLOGICAL SIGNAL COMMUNICATION TERMINAL, WIRELESS BIOLOGICAL SIGNAL COMMUNICATION SYSTEM, AND WIRELESS BIOLOGICAL SIGNAL MONITORING SYSTEM

(71) Applicants: National University Corporation Tokyo Medical and Dental University, Tokyo (JP); TDK CORPORATION, Tokyo (JP)

(72) Inventors: Shigenori Kawabata, Tokyo (JP); Shuta Ushio, Tokyo (JP); Tomohiko Shibuya, Tokyo (JP)

(73) Assignees: National University Corporation Tokyo Medical and Dental University, Tokyo (JP); TDK CORPORATION, Tokyo (JP)

(21) Appl. No.: 16/335,583

(22) PCT Filed: Sep. 22, 2017
(86) PCT No.: PCT/JP2017/034294

§ 371 Date: Mar. 21, 2019

(30) Foreign Application Priority Data
Sep. 23, 2016 (JP) 2016-186010

(51) Int. Cl. A61B 5/04; A61B 5/00; A61B 5/0456; A61B 5/0488

(52) U.S. Cl. CPC A61B 5/04001; A61B 5/04014; A61B 5/0004; A61B 5/7225; A61N 1/0456; A61B 5/0488; A61B 5/04017; A61B 2505/05; A61B 5/0456

(57) ABSTRACT

A wireless biological signal communication terminal is provided with: a sensor unit which detects a biological signal; an A/D converting unit which performs A/D conversion of the biological signal in accordance with a set sampling frequency to obtain biological signal data; a recording unit which records a plurality of items of A/D converted biological signal data; a control unit which processes the plurality of items of biological signal data recorded by the recording unit in a prescribed period of time; a wireless module unit and an antenna which wirelessly transmit the result of the processing performed by the control unit to an external device; and a power supply unit which supplies power to drive the devices.

### TECHNICAL FIELD

The present invention relates to a wireless biological signal communication terminal, a wireless biological signal communication system and a wireless biological signal monitoring system.

### BACKGROUND ART

When performing surgery of spine, spinal cord or another area a physician monitors spinal cord and nerve function to prevent spine and nerve failure resulting from the operation. During this monitoring, a monitoring device applies periodic electrical stimulation to the cranial of the patient a plurality of times (approximately three to ten times) in order to detect myoelectric potential of the limbs of the patient. Then, the monitoring device performs signal averaging processing on the myoelectric potential detected a plurality of times and displays a waveform (transcranial motor evoked potential) as the result of the averaging processing on a monitor. The physician confirms the waveform displayed on the monitor to make a diagnosis on spinal cord function.

In order to detect myoelectric potential, the physician needs to attach bioelectrodes to the patient's body and connect the bioelectrodes to an electromyograph using a lead. However, preparing to connect the bioelectrodes to the electromyograph takes a long time (for example, two people are required over one hour) because the wiring is complex. Attaching a large number of bioelectrodes to the body of a patient also increases the risk of infection, and hence strict supervision is required.

For analyzing activity in sports, a spontaneous myoelectric potential monitoring system with a wireless function is available on the market as a solution to the problem of complex wiring. However, the battery provided in this system runs out quickly and only allows for around two hours of continuous use. Batteries cannot be replaced or charged during continuous monitoring in surgery that can last up to eight hours. Therefore, it is difficult to use this system for monitoring nerves during surgery.

As one way to reduce power consumption, there is proposed an electrocardiographic signal detecting apparatus that includes an A/D converter for converting, into a digital signals, electrocardiographic signals detected through electrodes placed over the heart, a characteristic extraction part for extracting characteristics in the electrocardiographic signals converted by the A/D converter, a sampling control unit for changing the sampling frequency of the A/D converter on the basis of the characteristics in the electrocardiographic signals extracted by the characteristic extraction part, and a storage part for storing the electrocardiographic signals that were converted by the A/D converter (see Patent Document 1).

With this device, when the characteristic extraction part extracts certain characteristics from the electrocardiographic signals such as peak position, peak interval, peak level of the electrocardiographic signals and the like, the sampling control unit changes the sampling frequency according to those characteristics. Sampling is performed at a high sampling frequency for important characteristic portions at which the electrocardiographic signal changes and at a low sampling frequency for unnecessary characteristic portions. As a result, an accurate electrocardiogram waveform can be obtained with less data. Further, especially when using a battery-powered device, battery exhaustion can be minimized and detection can be performed over many hours.

Patent Document 1: Japanese Unexamined Patent Application, Publication No. 2010-094236

### DISCLOSURE OF THE INVENTION

### Problems to be Solved by the Invention

However, there is a limit as to how far power consumption can be reduced through merely changing sampling frequency. In other words, power consumption cannot be reduced to a satisfactory level through merely changing sampling frequency. During actual operation, many transmission/reception devices in wireless electrocardiographic monitors fail due to the battery failure in the transmission unit. More specifically, 15 accidents related to wireless electrocardiographic monitors have occurred in the past 10 years, with one third of those cases (five cases) reported as medical accidents that were caused by battery failure in a transmission device. As such, there is a need to prevent battery failure when biological signals are wirelessly communicated in a medical setting, and there is still room for improvement in terms of further reducing power consumption by reducing battery exhaustion using other approaches. Surgical monitoring that is safer and has shorter prep time can be implemented through combining a wireless function with a non-contact measurement technique using biomagnetism that does not require bioelectrodes to be attached to a patient.

It is an object of the present invention to provide a wireless biological signal communication terminal with which power consumption can be further reduced.

### Means for Solving the Problems

The inventors of the present invention conducted extensive study in order to solve the above-described problem and found that power consumption can be reduced to a minimum by, when biological signal data is processed by processing means, driving wireless transmission means so that the wireless transmission means wirelessly transmits results of the processing by the processing means to an external device, and, when the biological signal data has not yet been processed or is being processed by the processing means, not driving the wireless transmission means so that the wireless transmission means does not wirelessly transmit data to the external device. Thus, the present invention was completed. More specifically, the present invention provides the following.

(1) The present invention is a wireless biological signal communication terminal including: biological signal detection means for detecting a biological signal; A/D conversion means for converting the biological signal from analog to digital according to a set sampling frequency to generate biological signal data; storage means for storing a plurality of pieces of the biological signal data converted from analog to digital according to the sampling frequency; processing means for processing, within a predetermined period, the plurality of pieces of biological signal data that are stored in the storage means; wireless transmission means for wirelessly transmitting results of processing by the processing means to an external device; and power supply means for supplying power for driving the biological signal detection means, the A/D conversion means, the storage means, the processing means, and the wireless transmission means, the wireless transmission means being driven to wirelessly transmit results of the processing by the processing means to the external device when the biological signal data is processed by the processing means, and the wireless transmission means not being driven and not wirelessly transmitting data to the external device when the plurality of pieces of biological signal data are not yet processed or are currently being processed by the processing means.

(2) In addition, the present invention is the wireless biological signal communication terminal according to (1) in which the biological signal data is waveform data on the biological signal and the processing means performs signal averaging processing on a plurality of pieces of the waveform data to generate averaged waveform data.

(3) In addition, the present invention is the wireless biological signal communication terminal according to (1) or (2), further including switching means for switching the sampling frequency.

(4) In addition, the present invention is the wireless biological signal communication terminal according to any one of (1) to (3) in which the biological signal detection means includes an electric sensor, a magnetic sensor, an acceleration sensor, or any combination thereof.

(5) In addition, the present invention is a wireless biological signal communication system including the wireless biological signal communication terminal and the external device of any one of (1) to (4), the external device including: reception means for receiving results of processing by the processing means that are transmitted from the wireless transmission means; and display means for displaying the results received by the reception means.

(6) In addition, the present invention is a wireless biological signal monitoring system including: electrical stimulation generation means for generating periodic electrical stimulation a plurality of times in one cycle; biological signal detection means for detecting one biological signal for each of the plurality of times of electrical stimulation; A/D conversion means for converting the biological signals from analog to digital at each detection according to a set sampling frequency to generate a plurality of pieces of biological signal data; storage means for storing a plurality of pieces of the biological signal data converted to digital according to the sampling frequency; processing means for collectively processing the plurality of pieces of biological signal data stored in the storage means in one cycle of electrical stimulation; wireless transmission means for wirelessly transmitting results of processing by the processing means; reception means for receiving the results of processing by the processing means that are transmitted from the wireless transmission means; display means for displaying the results received by the reception means; and power supply means for supplying power for driving the biological signal detection means, the A/D conversion means, the storage means, the processing means, and the wireless transmission means, the power supply means supplying power to the wireless transmission means while the wireless transmission means wirelessly transmits the results of processing by the processing means, and not supplying power to the wireless transmission means during detection by the biological signal detection means, conversion by the A/D conversion means, storage by the storage means, and processing by the processing means.

### Effects of the Invention

According to the present invention, there can be provided a wireless biological signal communication terminal and a wireless biological signal communication system with which power consumption can be further reduced. There can also be provided a wireless biological signal monitoring system that ensures safety even when used for surgical monitoring in which biological signal data is wirelessly transmitted using a battery.

### BRIEF DESCRIPTION OF THE DRAWINGS

FIG. 1 is a block diagram for illustrating the configuration of a wireless biological signal communication terminal 1 according to an embodiment.

FIG. 2 is a diagram for schematically illustrating waveform data generated by an A/D converter 12.

FIG. 3 is a diagram for schematically illustrating a biological signal that is generated by using electrical stimulation on a patient.

### PREFERRED MODE FOR CARRYING OUT THE INVENTION

A specific embodiment of the present invention is described in detail below, but the present invention is not limited to the described embodiment and may be changed as necessary without departing from the scope of the present invention.

### <Wireless Biological Signal Communication Terminal 1>

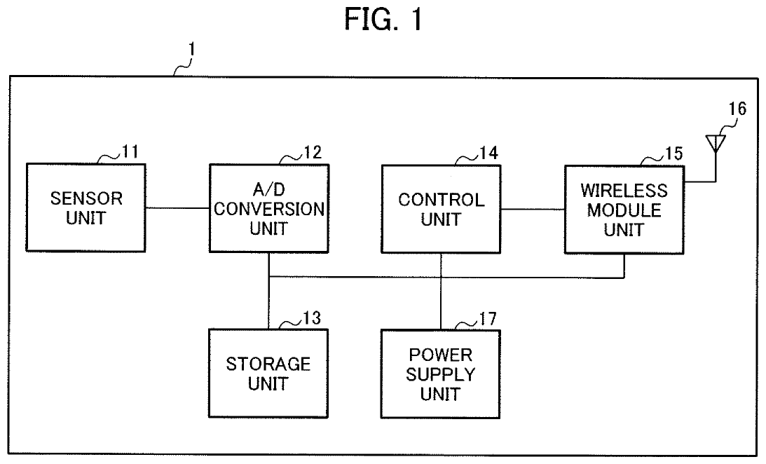

FIG. 1 is a block diagram for illustrating the configuration of a wireless biological signal communication terminal 1 according to this embodiment. The wireless biological signal communication terminal 1 includes a sensor unit 11 that functions as biological signal detection means for detecting biological signals, an A/D converter 12 that functions as A/D conversion means for converting the biological signals from analog to digital according to a set sampling frequency to generate biological signal data, a storage unit 13 that functions as storage means for storing a plurality of pieces of the biological signal data converted from analog to digital according to the sampling frequency, a control unit 14 that functions as processing means for processing, within a predetermined period, the plurality of pieces of biological signal data stored in the storage means, a wireless module unit 15 and an antenna 16 that function as wireless transmission means for wirelessly transmitting results of processing by the control unit 14 to an external device (not shown), and a power supply unit 17 that functions as power supply means to supply power for driving the sensor unit 11, the A/D converter 12, the storage unit 13, the control unit 14, and the wireless module unit 15.

When the control unit 14 processes the biological signal data, the wireless module unit 15 is driven to wirelessly transmit the results of processing by the control unit 14 to the external device. If the control unit 14 has not yet processed the plurality of pieces of biological signal data or is currently processing the biological signal data, the wireless module unit 15 is not driven and does not wirelessly transmit data to the external device.

### [Sensor Unit 11]

The sensor unit 11 is not particularly limited provided that the sensor unit 11 is a biological sensor. For example, the sensor unit 11 may be an electric sensor, a magnetic sensor, an acceleration sensor, a current sensor, an angular sensor, a piezoelectric sensor, or a combination of any of those sensors.

### [A/D Converter 12]

The A/D converter 12 converts analog signals (biological signals) that are output from the sensor unit 11 into digital signals (biological signal data) according to a set sampling frequency.

Although not shown, the wireless biological signal communication terminal 1 may include an amplifier that amplifies the biological signals detected by the sensor unit 11. In this case, the A/D converter 12 is configured to sample the biological signals amplified by the amplifier and convert these signals into digital signals at the set sampling frequency.

Although not shown, the wireless biological signal communication terminal 1 includes a clock. The clock generates a clock signal that serves as a base for the sampling frequency.

The set value for the sampling frequency is not particularly limited provided that waveform data can be appropriately generated from the target biological signals.

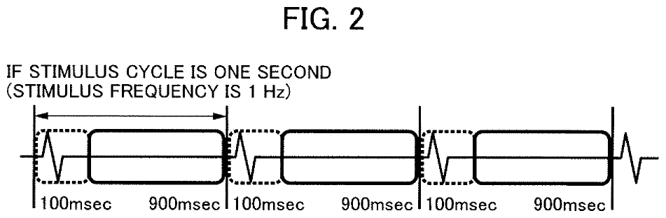

FIG. 2 is a diagram for schematically illustrating waveform data that is generated by the A/D converter 12 and corresponds to operation of the A/D converter 12 when periodic electrical stimulation is applied on a patient (in FIG. 2, the stimulus cycle is one second (stimulus frequency is 1 Hz)) as an example of monitoring spinal cord and nerve function. The horizontal axis in FIG. 2 represents time that passes after measurement of the biological signal starts. The vertical axis in FIG. 2 represents the magnitude of the set value for the target biological signal.

When the stimulus cycle is one second, the A/D converter 12 performs A/D conversion on the biological signals detected by the sensor unit 11 for only a period, for example 100 milliseconds, required for surgical monitoring during that one second. During the other 900 milliseconds, the A/D converter 12 does not perform A/D conversion. Every second, the A/D converter 12 iterates performing and not performing A/D conversion.

Therefore, as illustrated in FIG. 2, a waveform is present for 100 milliseconds within one second. During the other 900 milliseconds, no waveform is present.

In other words, when the stimulus cycle is one second, the A/D converter 12 only consumes a relatively large amount of power in the 100 millisecond period and, during the other 900 milliseconds, the A/D converter 12 hardly consumes any power. Therefore, generally speaking, power consumption required for surgical monitoring increases as the stimulus cycle becomes shorter and the period in which A/D conversion is performed becomes longer. Further, power consumption required for sampling increases as the sampling frequency increases and power consumption required to transmit waveform data on the generated biological signal increases as the amount of waveform data increases. In light of this, in order to reduce power consumption, the stimulus cycle is preferably set long, the A/D conversion period is preferably set short, and the sampling frequency is preferably set low within a range that does not impede on surgical monitoring.

### [Storage Unit 13]

The storage unit 13 stores programs that run processing for each unit in the wireless biological signal communication terminal 1, as well as a plurality of pieces of biological signal data that have been converted from analog to digital according to the set sampling frequency.

For example, as illustrated in FIG. 2, if electrical stimulation is sporadically applied at a stimulus cycle of one second (stimulus frequency of 1 Hz) and a biological signal is detected once within the cycle, biological signal data is stored once each second. While FIG. 2 has been simplified for ease of description, electrical stimulation is usually applied a plurality of times within one cycle, biological signals are detected each time electrical stimulation is applied, and biological signal data is stored a plurality of times.

### [Control Unit 14]

The control unit 14 functions as processing means. The control unit 14 runs processing for each unit in the wireless biological signal communication terminal 1 according to the programs stored in the storage unit 13. For example, the control unit 14 processes, within a predetermined period, the plurality of pieces of biological signal data stored in the storage unit 13.

The calculation processing method performed by the control unit 14 is not particularly limited and may be signal averaging processing, moving average processing, Wiener filter processing, low-pass filter (LPF) processing, high-pass filter (HPF) processing, band-pass filter (BPF) processing, or band elimination filter (BEF) processing. In particular, in order to more easily reduce noise such as environmental magnetism, the calculation processing method is preferably signal averaging processing in which a plurality of pieces of waveform data are averaged to generate averaged waveform data.

The length of the predetermined period in which the biological signal data is processed is not particularly limited and may be any length provided that calculation processing such as signal averaging processing, moving average processing or Wiener filter processing can be appropriately performed.

In terms of reducing power consumed by the power supply unit 17, the control unit 14 preferably performs processing on the plurality of types of biological signal data as little as possible within a range that does not hinder the accuracy of biological signal monitoring. In addition, the predetermined period for processing is preferably as short as possible.

### [Wireless Module Unit 15]

The wireless module unit 15 wirelessly transmits results of processing by the control unit 14 to an external device (not shown). The wireless module unit 15 includes a modulator that modulates the results of processing by the control unit 14 into radio signals, the antenna 16 that transmits the radio signals to the external device, and other components.

### [Power Supply Unit 17]

The power supply unit 17 is not particularly limited and may be primary battery, a secondary battery or another type of battery provided that the power supply unit 17 can supply power to the A/D converter 12, the storage unit 13, the control unit 14 and the wireless module unit 15. For example, a small and lightweight battery such as a lithium battery is preferably used as the power supply unit 17.

In this embodiment, when the control unit 14 processes the biological signal data, the wireless module unit 15 is driven to wirelessly transmit the results processed by the control unit 14 to the external device. On the other hand, when the control unit 14 does not yet processed the plurality of pieces of biological signal data or is currently processing the biological signal data, the wireless module unit 15 is not driven and does not wirelessly transmit data to the external device.

In a conventional wireless biological signal communication terminal, the wireless module unit constantly runs and externally transmits electrocardiogram signals and information on the sampling frequency. Therefore, as described in Patent Document 1, there is a limit to how far the power consumed by the power supply unit can be reduced, even if sampling is performed at a high sampling frequency for important characteristic portions at which the electrocardiogram signal changes and at a low sampling frequency for unnecessary characteristic portions.

The wireless biological signal communication terminal 1 according to this embodiment uses the control unit 14 to perform calculation processing on a plurality of pieces of biological signal data. After performing the calculation processing, the wireless module unit 15 compiles only the results of the calculation processing and wirelessly transmits those results to an external device. The wireless module unit 15 does not wirelessly transmit any data to the external device after the compiled data has been transmitted to reduce unnecessary power consumption. In other words, the wireless biological signal communication terminal 1 does not constantly transmit information.

According to this embodiment, there can be provided the wireless biological signal communication terminal 1 with which power consumption can be further reduced.

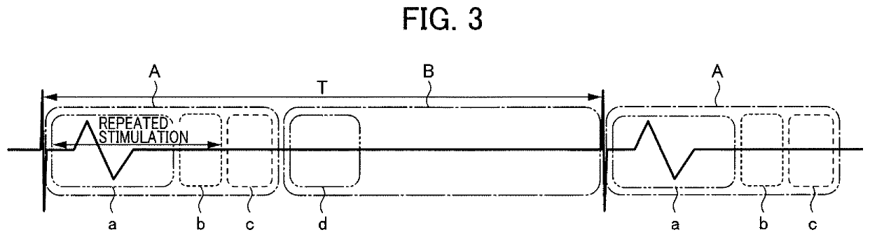

A detailed embodiment is described below with reference to FIG. 3. FIG. 3 schematically illustrates a biological signal obtained by using electrical stimulation on a patient. The horizontal axis in FIG. 3 represents time that passes after measurement of biological signals starts. The vertical axis in FIG. 3 represents the magnitude of the set value for the target biological signal. In this embodiment, as illustrated in FIG. 3, the stimulus cycle T is one second (stimulus frequency of 1 Hz), electrical stimulation is applied to a body a plurality of times within the stimulus cycle T, the first 100 milliseconds in the stimulus cycle T in which waveform data (biological signal data) on the biological signal and the like are acquired is defined as a measurement time A in which the sensor unit 11 and other components perform measurement, and the other 900 milliseconds are defined as a non-measurement time B.

During the measurement time A (100 milliseconds), the A/D converter 12 only converts the biological signal that is detected by the sensor unit 11 from analog to digital while acquiring the biological signal data necessary for surgical monitoring once for each stimulus cycle T (one second) and the storage unit 12 stores the biological signal data that has been converted to digital. A/D conversion and data storage are performed for each repeated electrical stimulation performed a plurality of times. Then, the control unit 14 performs signal averaging processing on the plurality of pieces of stored biological signal data. During the measurement time A, the biological signal data is not yet processed or is currently being processed and the wireless module unit 15 does not wirelessly transmit the biological signal data.

More specifically, the measurement time A includes a sample acquisition time a, a sample acquisition time b and a processing time c. The sample acquisition time a is a period where biological signals are to be detected, A/D conversion is to be performed and data is to be stored relative to portions at which signal averaging processing is performed, and is a stage at which the biological signal data is not yet processed. During the sample acquisition time a, the sampling frequency of the A/D converter 12 may be switched as necessary and, for example, the frequency can be made higher to enable more precise biological signal data acquisition. The sample acquisition time b is a period where biological signals are detected, A/D conversion is performed and data is stored, and is a stage at which the biological signal data is not yet processed. The sample acquisition time b is a time that occurs when the sample acquisition time a is made shorter than the iterated stimulation cycle. Because the signal averaging processing is not needed at this portion, the sample acquisition time b is preferably made as short as possible (in this embodiment, the sample acquisition time b is zero seconds). If the sample acquisition time b occurs, the sampling frequency for A/D conversion does not need to be increased. In FIG. 3, only one of each of the sample acquisition time a and the sample acquisition time b are shown, but the occurrences of the sample acquisition time a and the sample acquisition time b correspond to the occurrences of iterated stimulation.

The processing time c is a period in which signal averaging processing is performed on the plurality of pieces of biological signal data (plurality of pieces of biological signal data stored according to the occurrences of iterated stimulation) stored during the sample acquisition time a and the sample acquisition time b. The processing time c is a stage at which the biological signal data is processed.

The non-measurement time B includes a transmission time d. The transmission time d is a period in which the wireless module unit 15 wirelessly transmits the biological signal data averaged in the processing time c to an external device. The length of the transmission time d is dependent on the amount of biological signal data. The amount of biological signal data varies depending on the sample acquisition time a, the sample acquisition time b and the sampling frequency of A/D conversion.

As described above, if the stimulus cycle T is one second, power is not supplied to the wireless module unit 15 and the wireless module unit 15 does not perform wireless transmission during the measurement time A (100 milliseconds) consisting of the sample acquisition time a (stage at which the biological signal data is not yet processed), the sample acquisition time b (stage at which the biological signal data is not yet processed) and the processing time c (stage at which the biological signal data is processed). Even during the non-measurement time B, the wireless module unit 15 is only supplied with power and performs wireless transmission during the transmission time d after the biological signal data has been processed. As a result, startup time of the wireless module unit 15 can be minimized and unnecessary power consumption can be reduced.

Generally speaking, power consumption required for sampling increases as the sampling frequency set in the A/D converter 12 increases. Because of this, the value for the sampling frequency is preferably set as low as possible while still enabling favorable generation of waveform data from the target biological signals in order to reduce power consumption.

For example, for transcranial electrical motor evoked potential measurement in surgical monitoring, the sampling frequency needs to be set to around 5,000 Hz. On the other hand, for continuous electromyography monitoring (free-running EMG measurement), the sampling frequency needs to be set to around 1,000 Hz.

Therefore, the wireless biological signal communication terminal 1 preferably further includes a sampling frequency switching unit (not shown) that functions as switching means for switching the set value of the sampling frequency. Through employing the sampling frequency switching unit, the set value of the sampling frequency can be reduced when switching from transcranial electrical motor evoked potential measurement to continuous electromyography monitoring (free-running EMG measurement). As a result, the amount of power consumed by the A/D converter 12 can be reduced, the amount of biological signal data can be reduced, and the time in which the wireless module unit 15 is driven can be reduced. Therefore, the amount of power consumed by the power supply unit 17 can be further reduced.

### <Wireless Biological Signal Communication System>

A wireless biological signal communication system according to this embodiment includes the above-described wireless biological signal communication terminal 1 and an external device (not shown).

### [External Device]

Although not shown, the external device functions as receiving means and includes a reception unit that receives data transmitted from the wireless module unit 15 of the wireless biological signal communication terminal 1 via the antenna 16, a memory that stores the data received by the reception unit, a waveform restoration unit that restores waveforms on the basis of the data stored in the memory, and a display unit that displays an electrocardiogram waveform that has been restored by the waveform restoration unit on a display.

### <Wireless Biological Signal Monitoring System>

A wireless biological signal monitoring system according to this embodiment includes, in the above-described wireless biological signal communication system, electrical stimulation generation means for generating periodic electrical stimulation a plurality of times in one cycle. Therefore, during surgery on the spine, spinal cord or another area, a physician can diagnose the function of the spine or another area by checking the state of biological signal data that corresponds to the electrical stimulation.

### EFFECTS OF THE INVENTION

A system for monitoring spontaneous myoelectric potential with a wireless function is commercially available for analyzing sports activity. However, the battery in this system runs out quickly and only allows for around two hours of continuous use. Therefore, it is difficult to use this system for monitoring biological signals during surgery that requires up to eight hours of continuous monitoring and in which batteries cannot be replaced or charged.

According to this embodiment, the wireless biological signal communication terminal 1 can be continuously driven for a long time even if the power supply unit 17 is a small and lightweight commonly-used battery such as a lithium battery because the wireless module unit 15 is driven as infrequently as possible. Therefore, the invention described in the embodiment is particularly effective in unique environments that require periodic electrical stimulation to be applied on a patient a plurality of times once per several minutes, such as for monitoring spinal cord and nerve function during surgery. Further, interruption to wireless transmission due to battery failure can be prevented. If the invention is to be used for detecting biological signals using a non-contact method employing biological magnetism that does not require biological electrodes to be attached to the patient, safer surgical monitoring with a shorter setup time can be achieved through using the non-contact method with the wireless biological signal communication terminal 1 according to the embodiment. In addition, the present invention is versatile because the present invention can be used for sports movement analysis and other purposes.

### EXPLANATION OF REFERENCE NUMERALS

1 wireless biological signal communication terminal
11 sensor unit
12 A/D converter
13 storage unit
14 control unit
15 wireless module unit
16 antenna
17 power supply unit

1. A wireless biological signal communication terminal comprising:
biological signal detection means for detecting a biological signal;
A/D conversion means for converting the biological signal from analog to digital according to a set sampling frequency to generate biological signal data;
storage means for storing a plurality of pieces of the biological signal data converted from analog to digital according to the sampling frequency;
processing means for processing, within a predetermined period, the plurality of pieces of biological signal data that are stored in the storage means;
wireless transmission means for wirelessly transmitting results of processing by the processing means to an external device; and
power supply means for supplying power for driving the biological signal detection means, the A/D conversion means, the storage means, the processing means, and the wireless transmission means,
the wireless transmission means being driven to wirelessly transmit results of the processing by the processing means to the external device when the biological signal data is processed by the processing means, and
the wireless transmission means not being driven and not wirelessly transmitting data to the external device when the plurality of pieces of biological signal data are not yet processed or are currently being processed by the processing means.

2. The wireless biological signal communication terminal according to claim 1,
wherein the biological signal data is waveform data on the biological signal, and
wherein the processing means performs signal averaging processing on a plurality of pieces of the waveform data to generate averaged waveform data.

3. The wireless biological signal communication terminal according to claim 1, further including switching means for switching the sampling frequency.

4. The wireless biological signal communication terminal according to claim 1, wherein the biological signal detection means includes an electric sensor, a magnetic sensor, an acceleration sensor, or any combination thereof.

5. A wireless biological signal communication system comprising:
the wireless biological signal communication terminal and the external device of claim 1,
the external device including:
reception means for receiving results of processing by the processing means that are transmitted from the wireless transmission means; and
display means for displaying the results received by the reception means.

6. A wireless biological signal monitoring system comprising:
electrical stimulation generation means for generating periodic electrical stimulation a plurality of times in one cycle;
biological signal detection means for detecting one biological signal for each of the plurality of times of electrical stimulation;
A/D conversion means for converting the biological signals from analog to digital at each detection according to a set sampling frequency to generate a plurality of pieces of biological signal data;
storage means for storing a plurality of pieces of the biological signal data converted to digital according to the sampling frequency;
processing means for collectively processing the plurality of pieces of biological signal data stored in the storage means in one cycle of electrical stimulation;
wireless transmission means for wirelessly transmitting results of processing by the processing means;
reception means for receiving the results of processing by the processing means that are transmitted from the wireless transmission means;
display means for displaying the results received by the reception means; and
power supply means for supplying power for driving the biological signal detection means, the A/D conversion means, the storage means, the processing means, and the wireless transmission means,
the power supply means supplying power to the wireless transmission means while the wireless transmission means wirelessly transmits the results of processing by the processing means, and not supplying power to the wireless transmission means during detection by the biological signal detection means, conversion by the A/D conversion means, storage by the storage means, and processing by the processing means.

7. The wireless biological signal communication terminal according to claim 2, further including switching means for switching the sampling frequency.

8. The wireless biological signal communication terminal according to claim 2, wherein the biological signal detection means includes an electric sensor, a magnetic sensor, an acceleration sensor, or any combination thereof.

9. The wireless biological signal communication terminal according to claim 3, wherein the biological signal detection means includes an electric sensor, a magnetic sensor, an acceleration sensor, or any combination thereof.

10. A wireless biological signal communication system comprising:
the wireless biological signal communication terminal and the external device of claim 2,
the external device including:
reception means for receiving results of processing by the processing means that are transmitted from the wireless transmission means; and
display means for displaying the results received by the reception means.

11. A wireless biological signal communication system comprising:
the wireless biological signal communication terminal and the external device of claim 3,
the external device including:
reception means for receiving results of processing by the processing means that are transmitted from the wireless transmission means; and
display means for displaying the results received by the reception means.

12. A wireless biological signal communication system comprising:
the wireless biological signal communication terminal and the external device of claim 4,
the external device including:
reception means for receiving results of processing by the processing means that are transmitted from the wireless transmission means; and
display means for displaying the results received by the reception means.
